(19) United States
(12) Patent Application Publication
(10) Pub. No.: US 2022/0332706 A1
(43) Pub. Date: Oct. 20, 2022

(54) CRYSTALLINE FORMS OF A JAK2 INHIBITOR

(71) Applicant: Impact Biomedicines, Inc., Summit, NJ (US)

(72) Inventors: Benoit Robert, Paris (FR); Pascal Billot, Paris (FR)

(21) Appl. No.: 17/430,148

(22) PCT Filed: Feb. 11, 2020
(86) PCT No.: PCT/US20/17764

§ 371 Date: Aug. 11, 2021

(30) Foreign Application Priority Data
Feb. 12, 2019 (FR) FR1902018

(51) Int. Cl. C07D 403/12

(52) U.S. Cl. CPC C07D 403/12; C07B 2200/13

(57) ABSTRACT

The present disclosure provides crystalline forms of a JAK2 inhibitor, compositions thereof and methods of treating a JAK2-mediated disorder.

### CROSS-REFERENCE TO RELATED APPLICATIONS

The present application is the 371 national phase application of PCT Application No. PCT/US20/17764, filed Feb. 11, 2020, which claims priority to French application number FR1902018, filed Feb. 12, 2019, each of which is hereby incorporated by reference in its entirety.

### FIELD OF THE INVENTION

The present invention provides compounds, and compositions thereof, useful as inhibitors of protein kinases.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

In some embodiments, the present disclosure provides one or more crystalline forms of Compound 1:

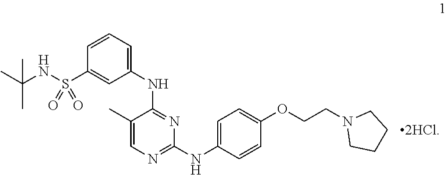 [SMILES: Cc1cnc(Nc2ccc(OCCN3CCCC3)cc2)nc1Nc1cccc(S(=O)(=O)NC(C)(C)C)c1]

In some embodiments, Compound 1 is useful in treating a myeloproliferative disorder. In some embodiments, a myeloproliferative disorder is selected from myelofibrosis, polycythemia vera and essential thrombocythemia. In some embodiments, myelofibrosis is selected from primary myelofibrosis or secondary myelofibrosis. In some embodiments, secondary myelofibrosis is selected from post-polycythemia vera and post-essential thrombocythemia.

In some embodiments, the present disclosure provides a method of inhibiting activity of a JAK2 kinase, or a mutant thereof, in a biological sample comprising the step of contacting said biological sample with Compound 1 (e.g., a crystalline anhydrous form, a crystalline hydrate form, a crystalline solvate form, or a crystalline heterosolvate form), or a composition thereof.

According to another embodiment, the present disclosure relates to a method of inhibiting activity of a JAK2 kinase, or a mutant thereof, in a patient comprising the step of administering to said patient Compound 1 (e.g., a crystalline anhydrous form, a crystalline hydrate form, a crystalline solvate form, or a crystalline heterosolvate form), or a composition thereof. In other embodiments, the present disclosure provides a method for treating a JAK2-mediated disease or disorder, in a patient in need thereof, comprising the step of administering to said patient Compound 1, or a composition thereof.

### BRIEF DESCRIPTION OF THE DRAWINGS

FIG. 1 depicts the FT-Raman spectrum of Form A of Compound 1.

FIG. 2 depicts the X-ray powder diffraction (XRPD) pattern of Form A of Compound 1.

FIG. 3A depicts the thermogravimetric analysis (TGA) pattern of Form A of Compound 1. FIG. 3B depicts the differential scanning calorimetry (DSC) pattern of Form A of Compound 1.

FIG. 4 depicts the FT-Raman spectrum of Form B of Compound 1.

FIG. 5 depicts the XRPD pattern of Form B of Compound 1.

FIG. 6A depicts the TGA pattern of Form B of Compound 1. FIG. 6B depicts the DSC pattern of Form B of Compound 1.

FIG. 7 depicts the XRPD pattern of Form C of Compound 1.

FIG. 8 depicts the XRPD pattern of Form D of Compound 1.

FIG. 9 depicts the XRPD pattern of Form E of Compound 1.

FIG. 10 depicts the XRPD pattern of Form F of Compound 1.

FIG. 11 depicts the TGA pattern of Form F of Compound 1.

FIG. 12 depicts the dynamic vapor sorption (DVS) isotherm of Form F of Compound 1.

FIG. 13 depicts the XRPD pattern of Form G of Compound 1.

FIG. 14 depicts the TGA pattern of Form G of Compound 1.

FIG. 15 depicts the DVS isotherm of Form G of Compound 1.

FIG. 16 depicts the XRPD pattern of Form H of Compound 1.

FIG. 17 depicts the TGA pattern of Form H of Compound 1.

FIG. 18 depicts the DVS isotherm of Form H of Compound 1.

FIG. 19 depicts the XRPD pattern of Form I of Compound 1.

### DETAILED DESCRIPTION OF THE INVENTION

### General Description of Certain Aspects of the Invention

U.S. Pat. No. 7,528,143, issued May 5, 2009 (“the '143 patent”), the entirety of which is hereby incorporated herein by reference, describes certain 2,4-disubstituted pyrimidine compounds that are useful in treating myeloproliferative disorders, including polycythemia vera, essential thrombocythemia and myelofibrosis (e.g., primary myelofibrosis and secondary myelofibrosis such as post-polycythemia vera myelofibrosis and post-essential thrombocythemia myelofibrosis). Such compounds include N-tert-butyl-3-[(5-methyl-2-{[4-(2-pyrrolidin-1-ylethoxy)phenyl]amino}pyrimidin-4-yl)amino]benzenesulfonamide:

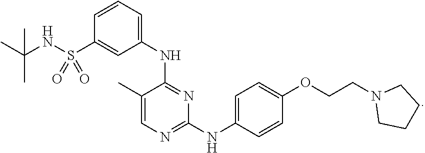 [SMILES: Cc1cnc(Nc2ccc(OCCN3CCCC3)cc2)nc1Nc1cccc(S(=O)(=O)NC(C)(C)C)c1]

N-tert-butyl-3-[(5-methyl-2-{[4-(2-pyrrolidin-1-ylethoxy)phenyl]amino}pyrimidin-4-yl)amino]benzenesulfonamide is designated as compound number LVII and the synthesis thereof is described in detail at Example 90 of the '143 patent.

N-tert-butyl-3-[(5-methyl-2-{[4-(2-pyrrolidin-1-ylethoxy)phenyl]amino}pyrimidin-4-yl)amino]benzenesulfonamide is active in a variety of assays and therapeutic models demonstrating inhibition of Janus kinase 2 (JAK2). Accordingly, N-tert-butyl-3-[(5-methyl-2-{[4-(2-pyrrolidin-1-ylethoxy)phenyl]amino}pyrimidin-4-yl)amino]benzenesulfonamide and salts, hydrates or solvates thereof are useful for treating one or more disorders associated with activity of JAK2.

In some embodiments, the present disclosure provides one or more crystalline forms of Compound 1:

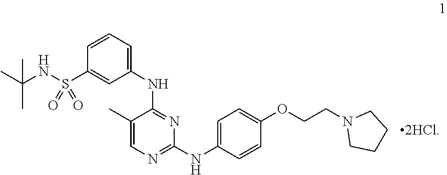 [SMILES: Cc1cnc(Nc2ccc(OCCN3CCCC3)cc2)nc1Nc1cccc(S(=O)(=O)NC(C)(C)C)c1]

It will be appreciated that a crystalline form of Compound 1 can exist in a neat or unsolvated form, a hydrated form, and/or a solvated form. In some embodiments, a crystalline form of Compound 1 is a neat or unsolvated crystal form and thus does not have any water or solvent incorporated into the crystal structure. In some embodiments, a crystalline form of Compound 1 is a hydrated or solvated form. In some embodiments, a crystalline form of Compound 1 is a hydrate/solvate form (also referred to herein as a “heterosolvate”).

Accordingly, in some embodiments, the present disclosure provides one or more crystalline anhydrous forms of Compound 1:

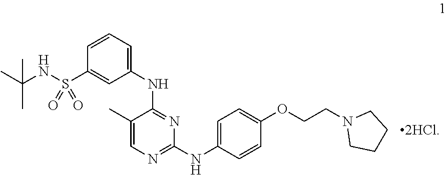 [SMILES: Cc1cnc(Nc2ccc(OCCN3CCCC3)cc2)nc1Nc1cccc(S(=O)(=O)NC(C)(C)C)c1]

In some embodiments, the present disclosure provides one or more crystalline hydrate forms of Compound 1:

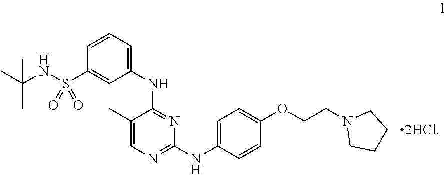 [SMILES: Cc1cnc(Nc2ccc(OCCN3CCCC3)cc2)nc1Nc1cccc(S(=O)(=O)NC(C)(C)C)c1]

In some embodiments, the present disclosure provides one or more crystalline solvate forms of Compound 1:

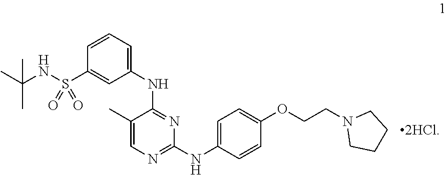 [SMILES: Cc1cnc(Nc2ccc(OCCN3CCCC3)cc2)nc1Nc1cccc(S(=O)(=O)NC(C)(C)C)c1]

In some embodiments, the present disclosure provides a sample comprising a crystalline form of Compound 1, wherein the sample is substantially free of impurities. As used herein, the term “substantially free of impurities” means that the sample contains no significant amount of extraneous matter. In some embodiments, a sample comprising a crystalline form of Compound 1 is substantially free of amorphous Compound 1. In certain embodiments, the sample comprises at least about 90% by weight of a crystalline form of Compound 1. In certain embodiments, the sample comprises at least about 91%, at least about 92%, at least about 93%, at least about 94% by weight of a crystalline form of Compound 1. In certain embodiments, the sample comprises at least about 95% by weight of a crystalline form of Compound 1. In still other embodiments, the sample comprises at least about 99% by weight of a crystalline form of Compound 1.

According to some embodiments, the sample comprises at least about 95, 97, 97.5, 98.0, 98.5, 99, 99.5, 99.8 weight percent (wt %) of a crystalline form of Compound 1, where the percentages are based on the total weight of the sample. According to some embodiments, a sample comprising a crystalline form of Compound 1 comprises no more than about 5.0 percent of total organic impurities. In some embodiments, a sample comprising a crystalline form of Compound 1 comprises no more than about 3.0 percent of total organic impurities. In some embodiments, a sample comprising a crystalline form of Compound 1 comprises no more than about 1.5 percent of total organic impurities. In some embodiments, a sample comprising a crystalline form of Compound 1 comprises no more than about 1.0 percent of total organic impurities. In some embodiments, a sample comprising a crystalline form of Compound 1 comprises no more than about 0.6 percent of total organic impurities. In some embodiments, a sample comprising a crystalline form of Compound 1 comprises no more than about 0.5 percent of total organic impurities. In some embodiments, the percent of total organic impurities is measured by HPLC.

It has been found that Compound 1 can exist in at least nine distinct crystal forms, or polymorphs.

In some embodiments, the present disclosure provides a crystalline hydrate form of Compound 1. In some such embodiments, a crystalline hydrate form of Compound 1 is a monohydrate. In some embodiments, a crystalline monohydrate form of Compound 1 is characterized by one or more peaks in its X-ray powder diffraction pattern selected from 4.3, 9.6, 10.0, 12.4, 12.7, and 17.0±0.2 degrees 2θ. In some such embodiments, a crystalline monohydrate form of Compound 1 is Form A.

In some embodiments, Form A is characterized by the following peaks in its X-ray powder diffraction pattern:

| Position | | |
|---|---|---|
| °2θ ± 0.2 | d-spacing | Height |
| degrees | [Å] | [cts] |
| 4.3 | 20.776 | 681 |
| 9.6 | 9.220 | 1040 |
| 10.0 | 8.883 | 692 |
| 12.4 | 7.163 | 825 |
| 12.7 | 6.944 | 1182 |
| 13.5 | 6.567 | 2426 |
| 14.2 | 6.226 | 1568 |
| 15.0 | 5.910 | 1612 |
| 16.0 | 5.531 | 2875 |
| 17.0 | 5.227 | 7509 |
| 17.6 | 5.051 | 2863 |
| 18.0 | 4.915 | 5744 |
| 18.6 | 4.776 | 1002 |
| 18.9 | 4.684 | 472 |
| 21.0 | 4.229 | 1594 |
| 21.7 | 4.089 | 3006 |
| 22.7 | 3.912 | 398 |
| 23.6 | 3.765 | 2011 |
| 24.1 | 3.695 | 7573 |
| 24.9 | 3.579 | 1015 |
| 25.3 | 3.520 | 2108 |
| 25.6 | 3.484 | 962 |
| 26.1 | 3.410 | 612 |
| 26.9 | 3.317 | 1246 |
| 27.2 | 3.282 | 1490 |
| 27.7 | 3.215 | 2008 |
| 30.0 | 2.979 | 962 |
| 30.5 | 2.934 | 727 |
| 31.0 | 2.888 | 431 |
| 31.9 | 2.803 | 508 |
| 34.3 | 2.615 | 915 |
| 35.1 | 2.557 | 556 |
| 36.3 | 2.475 | 517 |

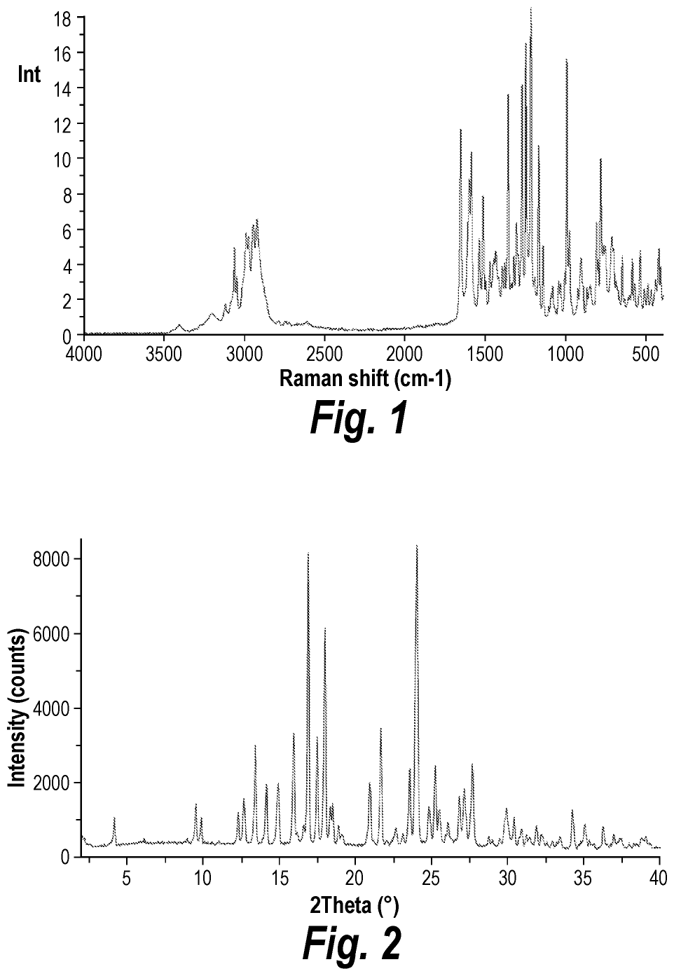

In some embodiments, Form A is characterized by the FT-Raman spectrum depicted in FIG. 1.

In some embodiments, Form A is characterized by the XRPD pattern depicted in FIG. 2.

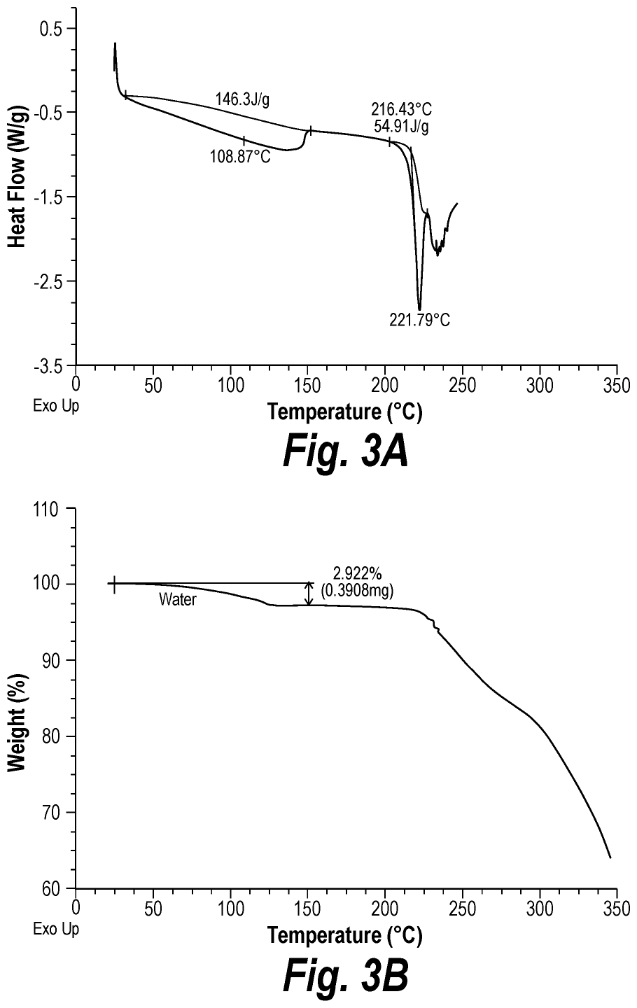

In some embodiments, Form A is characterized by the TGA pattern depicted in FIG. 3A. In some embodiments, Form A is characterized by the DSC pattern depicted in FIG. 3B.

In some embodiments, the present disclosure provides a crystalline trihydrate form of Compound 1. In some such embodiments, a crystalline trihydrate form of Compound 1 is characterized by one or more peaks in its X-ray powder diffraction pattern selected from 5.4, 6.2, 11.6, 13.9, 16.4, and 16.7±0.2 degrees 2θ. In some such embodiments, a crystalline trihydrate form of Compound 1 is Form B.

In some embodiments, Form B is characterized by the following peaks in its X-ray powder diffraction pattern:

| Position | | |
|---|---|---|
| °2θ ± 0.2 | d-spacing | Height |
| degrees | [Å] | [cts] |
| 5.4 | 16.435 | 988 |
| 6.2 | 14.334 | 494 |
| 6.9 | 12.755 | 169 |
| 10.4 | 8.487 | 191 |
| 11.6 | 7.640 | 607 |
| 13.4 | 6.631 | 845 |
| 13.9 | 6.373 | 1572 |
| 14.5 | 6.097 | 635 |
| 14.8 | 5.997 | 518 |
| 15.3 | 5.792 | 149 |
| 15.8 | 5.609 | 484 |
| 16.4 | 5.391 | 1340 |
| 16.7 | 5.299 | 1418 |
| 18.0 | 4.940 | 115 |
| 18.4 | 4.810 | 591 |
| 19.8 | 4.482 | 944 |
| 20.2 | 4.406 | 852 |
| 20.6 | 4.311 | 586 |
| 21.0 | 4.237 | 325 |
| 21.5 | 4.131 | 119 |
| 22.9 | 3.890 | 373 |
| 23.2 | 3.829 | 387 |
| 24.0 | 3.704 | 394 |
| 24.9 | 3.583 | 568 |
| 25.6 | 3.483 | 485 |
| 26.2 | 3.403 | 533 |
| 26.7 | 3.338 | 652 |
| 27.5 | 3.249 | 240 |
| 28.9 | 3.091 | 213 |
| 30.2 | 2.959 | 232 |
| 31.5 | 2.843 | 250 |
| 33.2 | 2.702 | 69 |
| 34.3 | 2.618 | 71 |
| 35.5 | 2.527 | 90 |

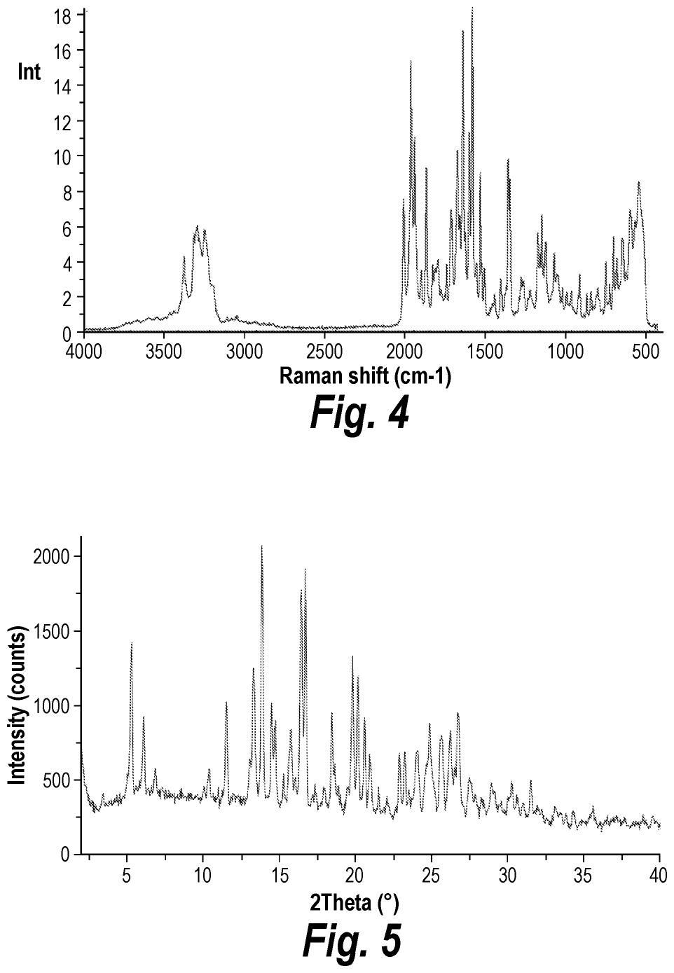

In some embodiments, Form B is characterized by the FT-Raman spectrum depicted in FIG. 4.

In some embodiments, Form B is characterized by the XRPD pattern depicted in FIG. 5.

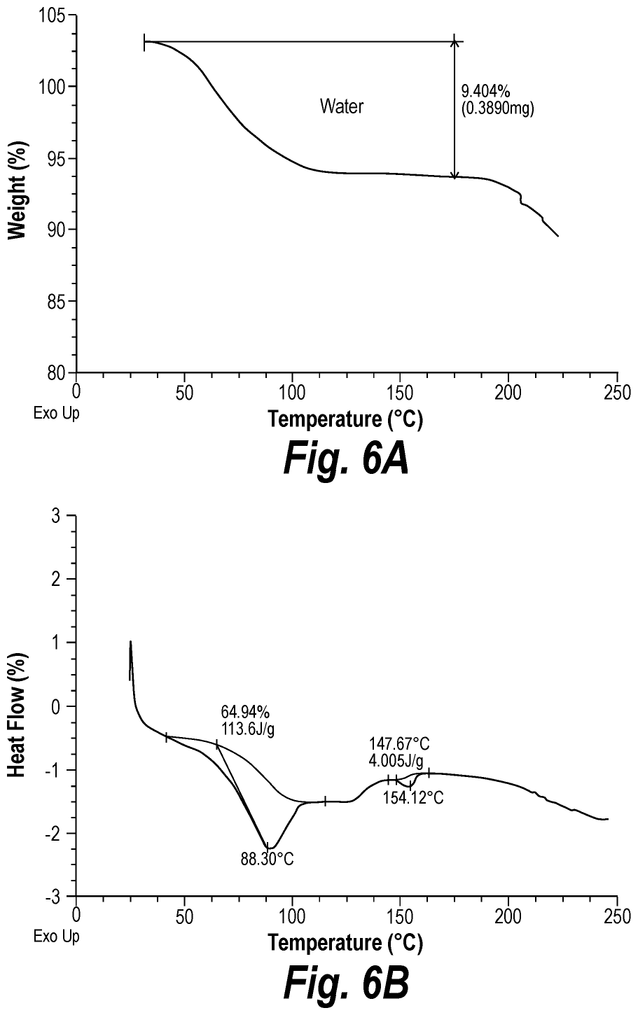

In some embodiments, Form B is characterized by the TGA pattern depicted in FIG. 6A. In some embodiments, Form B is characterized by the DSC pattern depicted in FIG. 6B.

In some embodiments, the present disclosure provides a crystalline anhydrous form of Compound 1. In some such embodiments, a crystalline anhydrous form of Compound 1 is characterized by one or more peaks in its X-ray powder diffraction pattern selected from 4.3, 6.2, 8.6, 9.7, 13.6, and 17.3±0.2 degrees 2θ. In some such embodiments, a crystalline anhydrous form of Compound 1 is Form C.

In some embodiments, Form C is characterized by the following peaks in its X-ray powder diffraction pattern:

Position
°2θ ± 0.2 degrees

4.3
6.2
8.6
9.7
10.0
12.3
13.1
13.6
14.0
15.2
15.8
16.4
17.3
17.7
18.0
18.6
19.8
20.7
21.8
22.0
22.2
23.7
24.3
24.8
25.4
25.7
25.9
26.1
26.9
27.4
27.7
28.3
29.2
29.5
35.1

In some embodiments, the present disclosure provides a method of preparing a crystalline anhydrous form of Compound 1 comprising heating, from about 40° C. to about 80° C., Form A under inert atmosphere. Accordingly, in some embodiments, the present disclosure provides a method of preparing Form C, the method comprising:

(a) providing Form A; and

(b) heating, from about 40° C. to about 80° C., Form A under inert atmosphere.

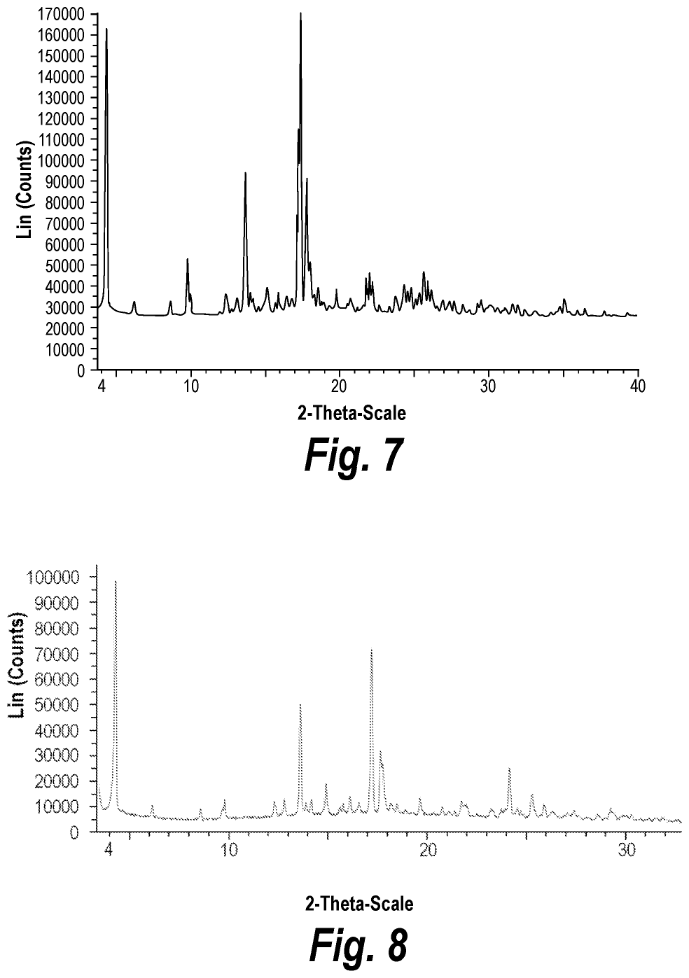

In some embodiments, Form C is characterized by the XRPD pattern depicted in FIG. 7.

In some embodiments, the present disclosure provides a crystalline anhydrous form of Compound 1 characterized by one or more peaks in its X-ray powder diffraction pattern selected from 12.8, 13.6, 14.9, 16.1, and 17.2±0.2 degrees 2θ. In some such embodiments, a crystalline anhydrous form of Compound 1 is Form D.

In some embodiments, Form D is characterized by the following peaks in its X-ray powder diffraction pattern:

Position
°2θ ± 0.2 degrees

4.3
6.1
8.5
9.8
12.3
12.8
13.6
13.9
14.1
14.9
16.1
17.2
17.7
19.6
21.8
21.9
24.2
25.3
25.9

In some embodiments, the present disclosure provides a method of preparing a crystalline anhydrous form of Compound 1 comprising heating, from about 25° C. to about 70° C., Form B under inert atmosphere. Accordingly, in some embodiments, the present disclosure provides a method of preparing Form D, the method comprising:

(c) providing Form B; and

(d) heating, from about 25° C. to about 70° C., Form B under inert atmosphere.

In some embodiments, Form D is characterized by the XRPD pattern depicted in FIG. 8.

In some embodiments, the present disclosure provides a crystalline solvate form of Compound 1. In some such embodiments, a crystalline solvate form of Compound 1 is a monosolvate. In some embodiments, a crystalline monosolvate form of Compound 1 is a monoisopropanol solvate. In some such embodiments, a crystalline monoisopropanol solvate form of Compound 1 is characterized by one or more peaks in its X-ray powder diffraction pattern selected from 7.2, 10.4, 10.8, 13.2, and 17.5±0.2 degrees 2θ. In some such embodiments, a crystalline monoisopropanol solvate form of Compound 1 is Form E.

In some embodiments, Form E is characterized by the following peaks in its X-ray powder diffraction pattern:

Position
°2θ ± 0.2 degrees

7.2
10.4
10.8
13.2
17.5
20.1
24.7
26.6
27.4

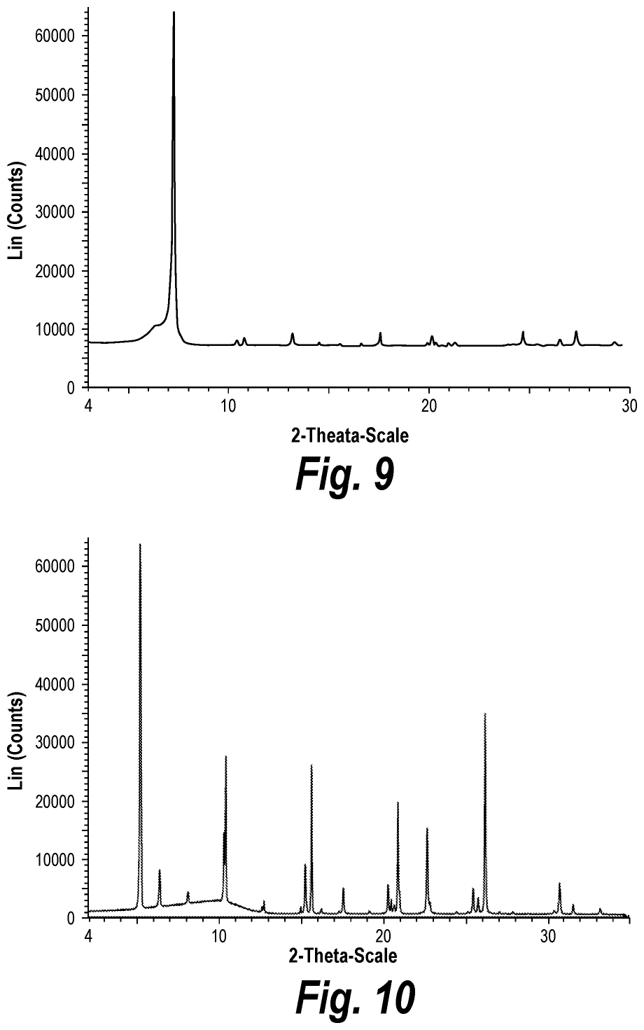

In some embodiments, Form E is characterized by the XRPD pattern depicted in FIG. 9.

In some embodiments, a crystalline solvate form of Compound 1 is a tetrasolvate. In some embodiments, a crystalline tetrasolvate form of Compound 1 is a tetraisopropanol solvate. In some embodiments, a crystalline tetraisopropanol solvate form of Compound 1 is characterized by one or more peaks in its X-ray powder diffraction pattern selected from 5.3, 6.4, 8.2, 10.5, 15.3, and 15.7±0.2 degrees 2θ. In some such embodiments, a crystalline tetraisopropanol solvate form of Compound 1 is Form F.

In some embodiments, Form F is characterized by the following peaks in its X-ray powder diffraction pattern:

Position
°2θ ± 0.2 degrees

5.3
6.4
8.2
10.5
15.3
15.7
17.7
20.4
21.0
22.8
25.6
25.9
26.4
31.0

In some embodiments, Form F is characterized by the XRPD pattern depicted in FIG. 10.

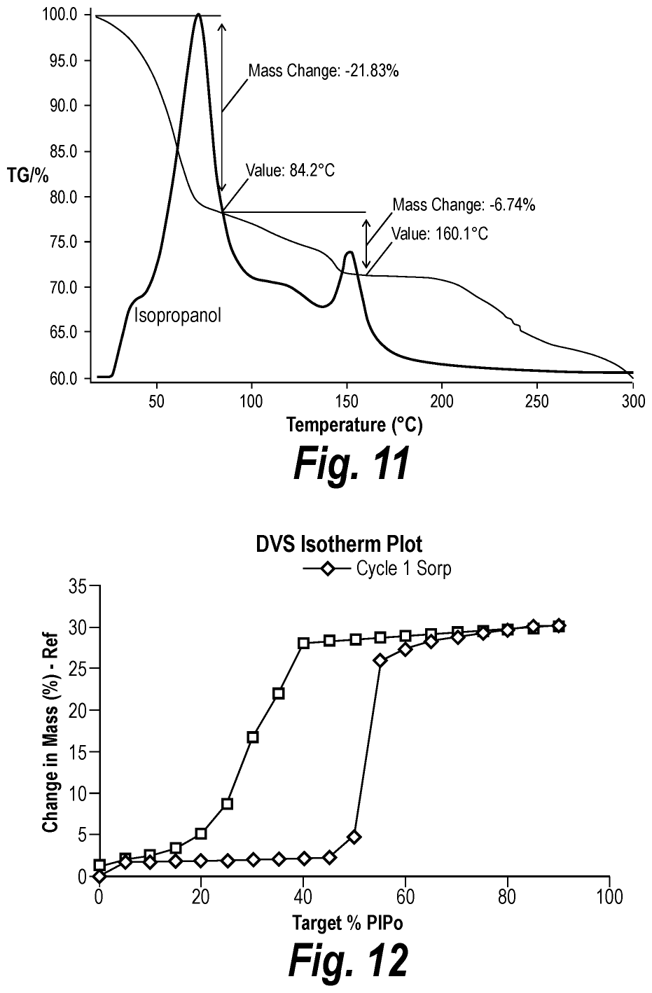

In some embodiments, Form F is characterized by the TGA pattern depicted in FIG. 11.

In some embodiments, Form F is characterized by the DVS isotherm depicted in FIG. 12.

In some embodiments, a crystalline solvate form of Compound 1 is a heterosolvate. In some embodiments, a crystalline heterosolvate form of Compound 1 is a water-isopropanol heterosolvate. In some embodiments, a crystalline water-isopropanol heterosolvate form of Compound 1 is characterized by one or more peaks in its X-ray powder diffraction pattern selected from 7.3, 7.6, 10.4, 10.8, and 17.5±0.2 degrees 2θ. In some such embodiments, a crystalline water-isopropanol heterosolvate form of Compound 1 is Form G.

In some embodiments, Form G is characterized by the following peaks in its X-ray powder diffraction pattern:

Position
°2θ ± 0.2 degrees

7.3
7.6
10.4
10.8
13.2
14.9
17.5
18.3
20.2
21.3
24.8
24.9
26.6
27.4
30.1

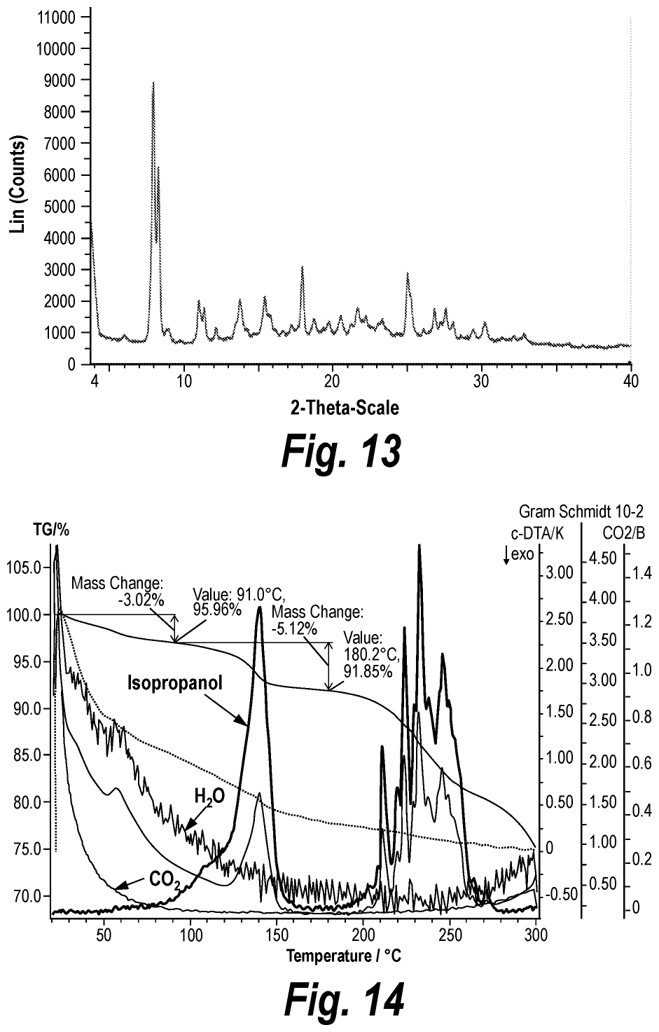

In some embodiments, Form G is characterized by the XRPD pattern depicted in FIG. 13.

In some embodiments, Form G is characterized by the TGA pattern depicted in FIG. 14.

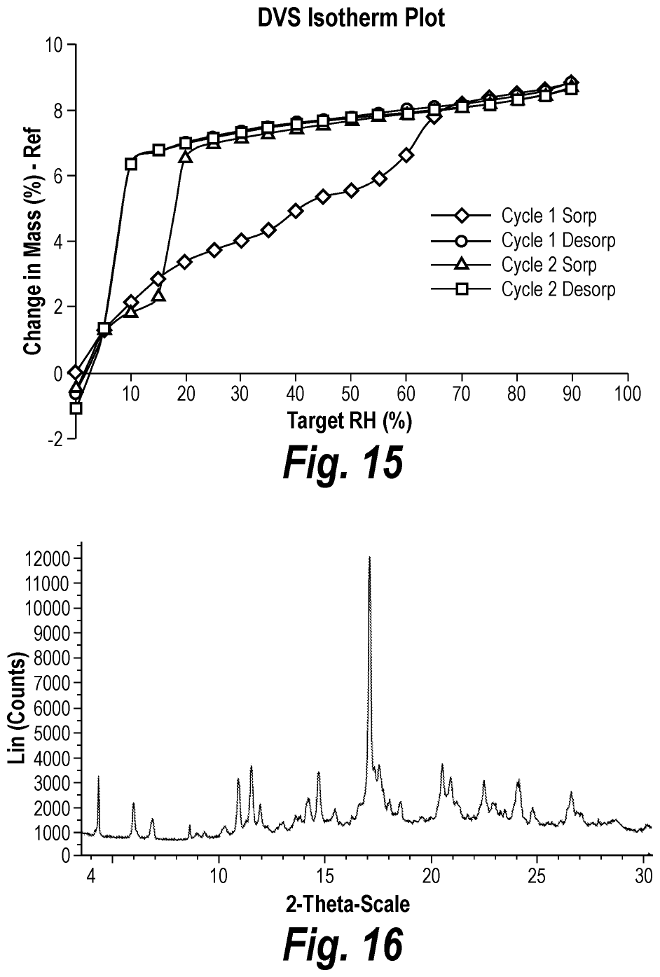

In some embodiments, Form G is characterized by the DVS isotherm depicted in FIG. 15.

In some embodiments, a crystalline solvate form of Compound 1 is a hexafluoroisopropanol solvate. In some embodiments, a crystalline hexafluoroisopropanol solvate form of Compound 1 is characterized by one or more peaks in its X-ray powder diffraction pattern selected from 4.3, 6.0, 6.9, 10.9, 11.5, 14.7, and 17.1±0.2 degrees 2θ. In some such embodiments, a crystalline hexafluoroisopropanol solvate form of Compound 1 is Form H.

In some embodiments, Form H is characterized by the following peaks in its X-ray powder diffraction pattern:

Position
°2θ ± 0.2 degrees

4.3
6.0
6.9
8.6
10.9
11.5
12.0
14.2
14.7
17.1
14.6
20.6
21.0
22.5
24.2
26.7
31.7

In some embodiments, Form H is characterized by the XRPD pattern depicted in FIG. 16.

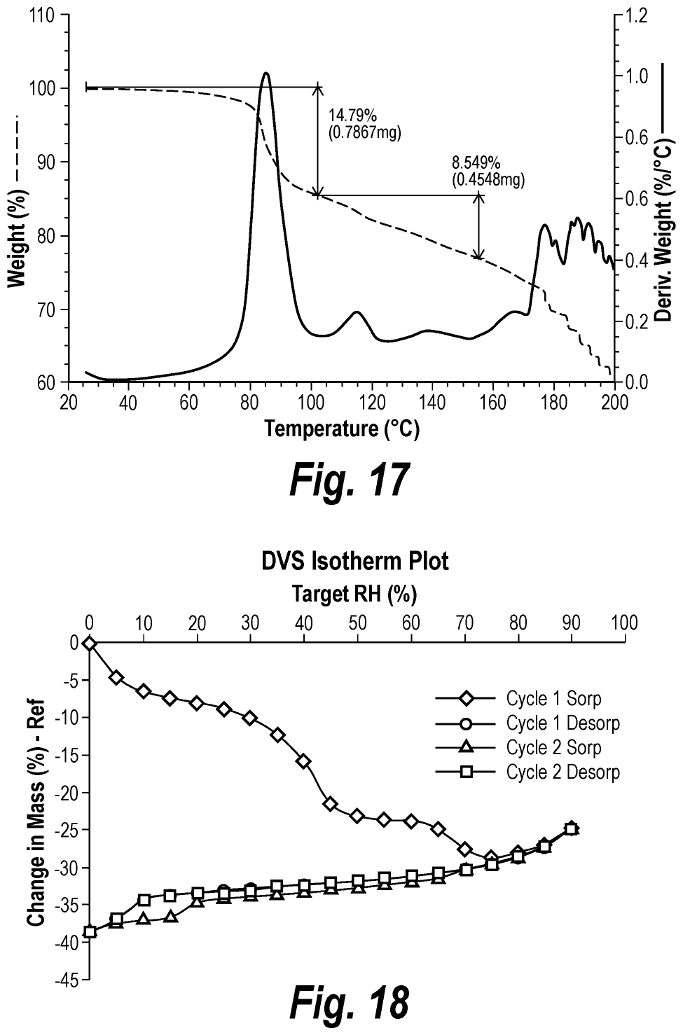

In some embodiments, Form H is characterized by the TGA pattern depicted in FIG. 17.

In some embodiments, Form H is characterized by the DVS isotherm depicted in FIG. 18.

In some embodiments, a crystalline solvate form of Compound 1 is an ethanol solvate. In some embodiments, a crystalline ethanol solvate form of Compound 1 is characterized by one or more peaks in its X-ray powder diffraction pattern selected from 5.3, 10.6, and 15.9±0.2 degrees 2θ. In some such embodiments, a crystalline ethanol solvate form of Compound 1 is Form I.

In some embodiments, Form I is characterized by the following peaks in its X-ray powder diffraction pattern:

Position
°2θ ± 0.2 degrees

5.3
10.6
15.9
21.2
26.6

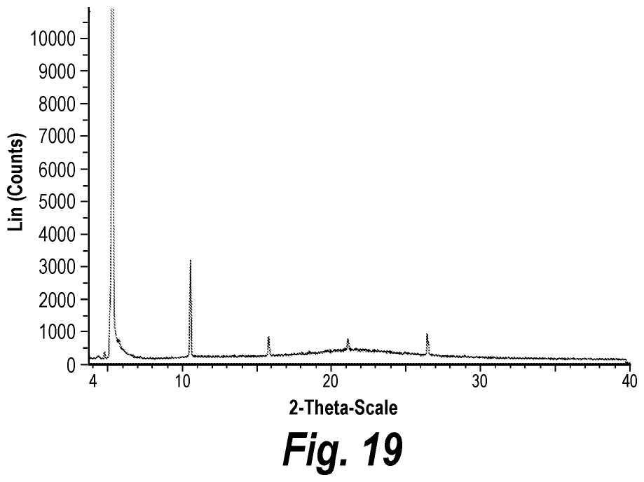

In some embodiments, Form I is characterized by the XRPD pattern depicted in FIG. 19.

### Uses, Formulation and Administration

Pharmaceutically Acceptable Compositions

According to another embodiment, the present disclosure provides a composition comprising Compound 1 (e.g., a crystalline anhydrous form, a crystalline hydrate form, a crystalline solvate form, or a crystalline heterosolvate form) and a pharmaceutically acceptable carrier, adjuvant, or vehicle. In certain embodiments, the amount of Compound 1 (e.g., a crystalline anhydrous form, a crystalline hydrate form, a crystalline solvate form, or a crystalline heterosolvate form) in compositions of this disclosure is such that it is effective to measurably inhibit JAK2, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compounds and compositions, according to method of the present invention, are administered using any amount and any route of administration effective for treating or lessening the severity of a disorder provided herein (i.e., a JAK2-mediated disease or disorder). The exact amount required will vary from subject to subject, depending on the species, age, and general condition of the subject, the severity of the infection, the particular agent, its mode of administration, and the like. Compound 1 (e.g., a crystalline anhydrous form, a crystalline hydrate form, a crystalline solvate form, or a crystalline heterosolvate form) is preferably formulated in unit dosage form for ease of administration and uniformity of dosage.

Compositions of the present disclosure may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of Compound 1 (e.g., a crystalline anhydrous form, a crystalline hydrate form, a crystalline solvate form, or a crystalline heterosolvate form), it is often desirable to slow the absorption of the compound from subcutaneous or intramuscular injection. This may be accomplished by the use of a liquid suspension of crystalline or amorphous material with poor water solubility. The rate of absorption of Compound 1 (e.g., a crystalline anhydrous form, a crystalline hydrate form, a crystalline solvate form, or a crystalline heterosolvate form) then depends upon its rate of dissolution that, in turn, may depend upon crystal size and crystalline form. Alternatively, delayed absorption of a parenterally administered Compound 1 (e.g., a crystalline anhydrous form, a crystalline hydrate form, a crystalline solvate form, or a crystalline heterosolvate form) is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping Compound 1 (e.g., a crystalline anhydrous form, a crystalline hydrate form, a crystalline solvate form, or a crystalline heterosolvate form) in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, Compound 1 (e.g., a crystalline anhydrous form, a crystalline hydrate form, a crystalline solvate form, or a crystalline heterosolvate form) is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compound 1 (e.g., a crystalline anhydrous form, a crystalline hydrate form, a crystalline solvate form, or a crystalline heterosolvate form) can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms Compound 1 (e.g., a crystalline anhydrous form, a crystalline hydrate form, a crystalline solvate form, or a crystalline heterosolvate form) may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to Compound 1 (e.g., a crystalline anhydrous form, a crystalline hydrate form, a crystalline solvate form, or a crystalline heterosolvate form), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions of this disclosure may be administered in the form of suppositories for rectal administration. These can be prepared by mixing Compound 1 (e.g., a crystalline anhydrous form, a crystalline hydrate form, a crystalline solvate form, or a crystalline heterosolvate form) with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing Compound 1 (e.g., a crystalline anhydrous form, a crystalline hydrate form, a crystalline solvate form, or a crystalline heterosolvate form) with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing Compound 1 (e.g., a crystalline anhydrous form, a crystalline hydrate form, a crystalline solvate form, or a crystalline heterosolvate form) suspended or dissolved in one or more carriers. Carriers for topical administration of Compound 1 (e.g., a crystalline anhydrous form, a crystalline hydrate form, a crystalline solvate form, or a crystalline heterosolvate form) include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing Compound 1 (e.g., a crystalline anhydrous form, a crystalline hydrate form, a crystalline solvate form, or a crystalline heterosolvate form) suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of Compound 1 (e.g., a crystalline anhydrous form, a crystalline hydrate form, a crystalline solvate form, or a crystalline heterosolvate form) include ointments, pastes, creams, lotions, gels, powders, solutions, sprays, inhalants or patches. Compound 1 (e.g., a crystalline anhydrous form, a crystalline hydrate form, a crystalline solvate form, or a crystalline heterosolvate form) is admixed under sterile conditions with a pharmaceutically acceptable carrier and any needed preservatives or buffers as may be required. Ophthalmic formulation, ear drops, and eye drops are also contemplated as being within the scope of this invention. Additionally, the present disclosure contemplates the use of transdermal patches, which have the added advantage of providing controlled delivery of Compound 1 (e.g., a crystalline anhydrous form, a crystalline hydrate form, a crystalline solvate form, or a crystalline heterosolvate form) to the body. Such dosage forms can be made by dissolving or dispensing the compound in the proper medium. Absorption enhancers can also be used to increase the flux of Compound 1 (e.g., a crystalline anhydrous form, a crystalline hydrate form, a crystalline solvate form, or a crystalline heterosolvate form) across the skin. The rate can be controlled by either providing a rate controlling membrane or by dispersing Compound 1 (e.g., a crystalline anhydrous form, a crystalline hydrate form, a crystalline solvate form, or a crystalline heterosolvate form) in a polymer matrix or gel.

In some embodiments, compositions described herein comprise an amount of Compound 1 (e.g., a crystalline anhydrous form, a crystalline hydrate form, a crystalline solvate form, or a crystalline heterosolvate form) that is the molar equivalent to free base N-tert-butyl-3-[(5-methyl-2-{[4-(2-pyrrolidin-1-ylethoxy)phenyl]amino}pyrimidin-4-yl)amino]benzenesulfonamide. For example, a 100 mg formulation of N-tert-butyl-3-[(5-methyl-2-{[4-(2-pyrrolidin-1-ylethoxy)phenyl]amino}pyrimidin-4-yl)amino]benzenesulfonamide (e.g., the unsolvated free base parent of Compound 1, MW=524.26) comprises 117.30 mg of Form A (MW=614.22).

In some embodiments, the present disclosure provides a composition comprising Compound 1 (e.g., a crystalline anhydrous form, a crystalline hydrate form, a crystalline solvate form, or a crystalline heterosolvate form), and one or more pharmaceutically acceptable excipients. In some embodiments, the one or more pharmaceutically acceptable excipients are selected from a binder and a lubricant.

In some embodiments, the binder is a microcrystalline cellulose. In some such embodiments, the microcrystalline cellulose is silicified microcrystalline cellulose.

In some embodiments, the binder is sodium stearyl fumarate.

In some embodiments, the composition comprises:

| Component | Amount | | Percentage |
|---|---|---|---|
| N-tert-butyl-3-[(5-methyl-2-{[4-(2-pyrrolidin-1- | 100 | mg | 35.53 |
| ylethoxy)phenyl]amino}pyrimidin-4- | | | |
| yl)amino]benzenesulfonamide | | | |
| silicified microcrystalline cellulose | 178.45 | mg | 63.404 |
| (high density 90 μm) | | | |
| sodium stearyl fumarate | 3.0 | mg | 1.066 |
| TOTAL | 281.45 | mg | 100 |

In certain embodiments, the composition comprises:

| Component | Amount | |
|---|---|---|
| Form A | 117.30 mg | |
| | (100 mg parent free base) | |
| silicified microcrystalline cellulose | 178.45 | mg |
| (high density 90 μm) | | |
| sodium stearyl fumarate | 3.0 | mg |
| TOTAL | 298.75 | mg |

### Uses of Compounds and Pharmaceutically Acceptable Compositions

Compounds and compositions described herein are generally useful for the inhibition of kinase activity of one or more enzymes. Examples of kinases that are inhibited by the compounds and compositions described herein and against which the methods described herein are useful include JAK2, or a mutant thereof.

The activity of Compound 1 (e.g., a crystalline anhydrous form, a crystalline hydrate form, a crystalline solvate form, or a crystalline heterosolvate form) utilized as an inhibitor of a JAK2 kinase, or a mutant thereof, may be assayed in vitro, in vivo or in a cell line. In vitro assays include assays that determine inhibition of either the phosphorylation activity and/or the subsequent functional consequences, or ATPase activity of activated JAK2 kinase, or a mutant thereof.

According to one embodiment, the invention relates to a method of inhibiting protein kinase activity in a biological sample comprising the step of contacting said biological sample with Compound 1 (e.g., a crystalline anhydrous form, a crystalline hydrate form, a crystalline solvate form, or a crystalline heterosolvate form), or a composition thereof.

According to another embodiment, the invention relates to a method of inhibiting activity of a JAK2 kinase, or a mutant thereof, in a biological sample comprising the step of contacting said biological sample with Compound 1 (e.g., a crystalline anhydrous form, a crystalline hydrate form, a crystalline solvate form, or a crystalline heterosolvate form), or a composition thereof.

According to another embodiment, the invention relates to a method of inhibiting activity of a JAK2 kinase, or a mutant thereof, in a patient comprising the step of administering to said patient Compound 1 (e.g., a crystalline anhydrous form, a crystalline hydrate form, a crystalline solvate form, or a crystalline heterosolvate form), or a composition thereof. In other embodiments, the present disclosure provides a method for treating a JAK2-mediated disease or disorder, in a patient in need thereof, comprising the step of administering to said patient Compound 1 (e.g., a crystalline anhydrous form, a crystalline hydrate form, a crystalline solvate form, or a crystalline heterosolvate form), or a pharmaceutically acceptable composition thereof. Such disorders are described in detail herein.

Crystal forms described herein are useful in treating a variety of disorders, including, but not limited to, for example, myeloproliferative disorders, proliferative diabetic retinopathy and other angiogenic-associated disorders including solid tumors and other types of cancer, eye disease, inflammation, psoriasis, and a viral infection. The kinds of cancer that can be treated include, but are not limited to, an alimentary/gastrointestinal tract cancer, colon cancer, liver cancer, skin cancer, breast cancer, ovarian cancer, prostate cancer, lymphoma, leukemia (including acute myelogenous leukemia and chronic myelogenous leukemia), kidney cancer, lung cancer, muscle cancer, bone cancer, bladder cancer or brain cancer.

Some examples of the diseases and disorders that can be treated also include ocular neovasculariaztion, infantile haemangiomas; organ hypoxia, vascular hyperplasia, organ transplant rejection, lupus, multiple sclerosis, rheumatoid arthritis, psoriasis, Type 1 diabetes and complications from diabetes, inflammatory disease, acute pancreatitis, chronic pancreatitis, asthma, allergies, adult respiratory distress syndrome, cardiovascular disease, liver disease, other blood disorders, asthma, rhinitis, atopic, dermatitits, autoimmune thryroid disorders, ulerative colitis, Crohn's disease, metastatic melanoma, Kaposi's sarcoma, multiple myeloma, conditions associated with cytokines, and other autoimmune diseases including glomerulonephritis, scleroderma, chronic thyroiditis, Graves' disease, autoimmune gastritis, autoimmune hemolytic anemia, autoimmune neutropenia, thrombocytopenia, atopy (e.g., allergic asthma, atopic dermatitis, or allergic rhinitis), chronic active hepatitis, myasthenia graivs, multiple scleroiss, inflammatory bowel disease, graft vs host disease, neurodegenerative diseases including motor neuron disease, Alzheimer's disease, Parkinson's disease, amyotrophic lateral scelerosis, Huntington's disease, cerebral ischemia, or neurodegenerative disease caused by traumatic injury, strike, gluatamate neurtoxicity or hypoxia; ischemic/reperfusion injury in stroke, myocardial ischemica, renal ischemia, heart attacks, cardiac hypertrophy, atherosclerosis and arteriosclerosis, organ hyoxia, and platelet aggregation.

Examples of some additional diseases and disorders that can be treated also include cell mediated hypersensitivity (allergic contact dermatitis, hypersensitivity pneumonitis), rheumatic diseases (e.g., systemic lupus erythematosus (SLE), juvenile arthritis, Sjogren's Syndrome, scleroderma, polymyositis, ankylosing spondylitis, psoriatic arthritis), viral diseases (Epstein Barr Virus, Hepatitis B, Hepatitis C, HIV, HTLVI, Vaicella-Zoster Virus, Human Papilloma Virus), food allergy, cutaneous inflammation, and immune suppression induced by solid tumors.

In some embodiments, Compound 1 (e.g., a crystalline anhydrous form, a crystalline hydrate form, a crystalline solvate form, or a crystalline heterosolvate form) is useful in treating a treating a myeloproliferative disorder. In some embodiments, the myeloproliferative disorder is selected from primary myelofibrosis, polycythemia vera, and essential thrombocythemia. In some embodiments, the myeloproliferative disorder is selected from primary myelofibrosis and secondary myelofibrosis. In some embodiments, the myeloproliferative disorder is secondary myelofibrosis. In some such embodiments, the secondary myelofibrosis is selected from post-polycythemia vera myelofibrosis and post-essential thrombocythemia myelofibrosis.

In some embodiments, a provided method comprises administering Compound 1 (e.g., a crystalline anhydrous form, a crystalline hydrate form, a crystalline solvate form, or a crystalline heterosolvate form) to a patient previously treated with a JAK2 inhibitor. In some such embodiments, a provided method comprises administering Compound 1 (e.g., a crystalline anhydrous form, a crystalline hydrate form, a crystalline solvate form, or a crystalline heterosolvate form) to a patient previously treated with ruxolitinib (JAKAFI®).

In some embodiments, a provided method comprises administering Compound 1 (e.g., a crystalline anhydrous form, a crystalline hydrate form, a crystalline solvate form, or a crystalline heterosolvate form) to a patient suffering from or diagnosed with a myeloproliferative disorder that is unresponsive to ruxolitinib. In some embodiments, the patient is suffering from or has been diagnosed with a myeloproliferative disorder that is refractory or resistant to ruxolitinib.

In some embodiments, the patient has relapsed during or following ruxolitinib therapy.

In some embodiments, the patient is intolerant to ruxolitinib. In some embodiments, patient intolerance to ruxolitinib is evidenced by a hematological toxicity (e.g., anemia, thrombocytopenia, etc.) or a non-hematological toxicity.

In some embodiments, the patient has had an inadequate response to or is intolerant to hydroxyurea.

In some embodiments, the patient is exhibiting or experiencing, or has exhibited or experienced, one or more of the following during treatment with ruxolitinib: lack of response, disease progression, or loss of response at any time during ruxolitinib treatment. In some embodiments, disease progression is evidenced by an increase in spleen size during ruxolitinib treatment.

In some embodiments, a patient previously treated with ruxolitinib has a somatic mutation or clonal marker associated with or indicative of a myeloproliferative disorder. In some embodiments, the somatic mutation is selected from a JAK2 mutation, a CALR mutation or a MPL mutation. In some embodiments, the JAK2 mutation is V617F. In some embodiments, the CALR mutation is a mutation in exon 9. In some embodiments, the MPL mutation is selected from W515K and W515L.

In some embodiments, the present disclosure provides a method of treating a relapsed or refractory myeloproliferative disorder, wherein the myeloproliferative disorder is relapsed or refractory to ruxolitinib.

In some embodiments, a myeloproliferative disorder is selected from intermediate risk myelofibrosis and high risk myelofibrosis.

In some embodiments, the intermediate risk myelofibrosis is selected from primary myelofibrosis, post-polycythemia vera (post-PV) myelofibrosis and post-essential thrombocythemia (post-ET) myelofibrosis. In some embodiments, the myelofibrosis is intermediate risk 1 (also referred to as intermediate-1 risk). In some embodiments, the myelofibrosis is intermediate risk 2 (also referred to as intermediate-2 risk).

In some embodiments, the high risk myelofibrosis is selected from primary myelofibrosis, post-polycythemia vera (post-PV) myelofibrosis and post-essential thrombocythemia (post-ET) myelofibrosis.

In some embodiments, the present disclosure provides an article of manufacture comprising a packaging material and a pharmaceutical composition contained within the packaging material. In some embodiments, the packaging material comprises a label which indicates that the pharmaceutical composition can be used for treatment of one or more disorders identified above.

### Example 1. Solubility Assessment

Solubility was assessed in an array of diverse solvents in order to facilitate the selection of solvent systems and corresponding dosing strategies for the subsequent crystal-form screening experiments. The solubility of Form A was visually estimated in 12 solvents at RT and, if applicable, at 40° C. by dosing small aliquots of the solvent into a fixed amount of the API (10.0 mg) until the dissolution point or a maximum volume of 1.8 mL was reached. As shown in Table 1, Form A exhibits high solubility (>100 mg/mL) in DMSO, MeOH, and water, but low solubility (≤5 mg/mL) in all other solvents evaluated.

TABLE 1
Estimated Solubility of Form A in 12 Solvents at RT and 40° C.

| | | Solubility at | Solubility at |
|---|---|---|---|
| # | Solvent (v/v) | RT [mg/mL] | 40° C. [mg/mL] |
| 1 | DMSO | >400 | N/A |
| 2 | MeOH | >400 | N/A |
| 3 | Water | 100-400 | N/A |
| 4 | IPA:water (9:1) | <5 | >5 |
| 5 | IPA | <5 | <5 |
| 6 | EtOAc | <5 | <5 |
| 7 | IPE | <5 | <5 |
| 8 | Toluene | <5 | <5 |
| 9 | THF | <5 | <5 |
| 10 | MeCN | <5 | <5 |
| 11 | MTBE | <5 | <5 |
| 12 | DCM | <5 | <5 |

N/A—Not applicable

### Example 2. Crystal Form Screen

FT-Raman Spectroscopy. Raman spectra were collected with a Nicolet NXR9650 or NXR 960 spectrometer (Thermo Electron) equipped with 1064 nm Nd:YVO₄ excitation laser, InGaAs and liquid-N₂ cooled Ge detectors, and a MicroStage. All spectra were acquired at 4 cm⁻¹ resolution, 64 scans, using Happ-Genzel apodization function and 2-level zero-filling through a glass cover.

Powder X-Ray Diffraction (PXRD). PXRD diffractograms were acquired on PANalytical X'Pert Pro diffractometer using Ni-filtered Cu Ka (45 kV/40 mA) radiation and a step size of 0.02° 20 and X'celerator™ RTMS (Real Time Multi-Strip) detector. Configuration on the incidental beam side: fixed divergence slit)(0.25°), 0.04 rad Soller slits, anti-scatter slit) (0.25°), and 10 mm beam mask. Configuration on the diffracted beam side: fixed divergence slit) (0.25°) and 0.04 rad Soller slit.

Differential Scanning calorimetry (DSC). DSC was conducted with a TA Instruments Q100 differential scanning calorimeter equipped with an autosampler and a refrigerated cooling system under 40 mL/min N₂ purge. DSC thermograms were obtained in crimped Al pans at 15° C./min in Al pans, unless noted otherwise.

Thermogravimetric Analysis (TGA). TGA thermograms were obtained with a TA Instruments Q500 thermogravimetric analyzer under 40 mL/min N₂ purge at 15° C./min in Al pans, unless noted otherwise.

Thermogravimetric Analysis with IR Off-Gas Detection (TGA-IR). TGA-IR was conducted with a TA Instruments Q5000 thermogravimetric analyzer interfaced to a Nicolet 6700 FT-IR spectrometer (Thermo Electron) equipped with an external TGA-IR module with a gas flow cell and DTGS detector. TGA was conducted with 60 mL/min N₂ flow and heating rate of 15° C./min in Pt or Al pans, unless noted otherwise. IR spectra were collected at 4 cm⁻¹ resolution and 32 scans at each time point.

Modulated Differential Scanning calorimetry (mDSC). mDSC was conducted with a TA Instruments Q200 differential scanning calorimeter equipped with an autosampler and a refrigerated cooling system under 40 mL/min N₂ purge. mDSC thermograms were obtained using modulation +/−0.32° C. every 60 seconds, and isothermal hold for 5.00 min, then a ramp 2.00° C./min to 200° C. Sample preparation was in crimped Al pans.

Lyophilization: Lyophilization was carried out on a Virtis Lyo-Centre Benchtop 3.5DBTZL (serial number: 41712). The unit was operated with a pressure of <10 mtorr and condenser temperature of <−100° C.

Ion Chromatography (IC). Ion chromatography was performed on a Dionex ICS-3000. Column: Dionex IonPac AS12A 4×200mm; Detection: Suppressed conductivity, ASRS 300 with suppressor current at 22 mA; Eluent (2.7 mM Na₂CO₃/0.3 mM NaHCO₃) at 1.5 mL/min.

Solvent Selection. The crystal form screen involved 48 solvent systems. The solvents were utilized as neat and binary mixtures to provide a diverse set of polarities, dielectric constants, dipole moments, and hydrogen-bond donor/acceptor attributes. Water-containing solvents with a variety of water activities were also included.

Crystallization Modes. The crystal form screening study employed the following crystallization modes using amorphous input material:
(1) Stirring suspensions while cycling the temperature between 45-5° C. for three days (TC, n=48)
(2) Cooling clarified solutions from 45° C. to 5° C., followed by a hold for four days (RC, n=48)
(3) Slow evaporation of solvents from solutions at RT over 7-10 days (EV, n=48; Form A input).

Analysis of Screening Products. FT-Raman spectroscopy was chosen as the primary method for analysis and grouping of samples. Representative samples from the groupings were analyzed by PXRD to verify their uniqueness. Where possible/practical, a representative sample of the unique form was further characterized by polarized-light microscopy, DSC, and/or TGA-IR.

Results of Screen. Form A was observed as the predominant output of the screening experiments. Form B, a hydrated form, was observed in 15 different slurry and evaporative experiments. As shown in Table 2, the crystal form screen of Compound 1 produced the following forms:

TABLE 2
Preliminary Crystal Form Screen

| | | | | | | Water |
|---|---|---|---|---|---|---|
| Solvent | TC | | RC | | EV | Activity |
| Water | NS | | NS | | NS | 1.00 |
| Methanol | NS | | NS | | B | |
| 2-Methoxyethanol | NS | | NS | | NS | |
| 1-Propanol | NS | | A | | A | |
| Nitromethane | A | | NS | | A | |
| Acetonitrile | A | | NS | | amorphous | |
| Dimethylsulfoxide | NS | | NS | | NS | |
| Acetone | A | | NS | | NS | |
| 2-Butanone | A | | NS | | NS | |
| Dichloromethane | A | B | A | FB | NS | |

| | | | | |
|---|---|---|---|---|
| Methyl acetate | A | NS | amorphous | |
| 4-Methyl-2-pentanone | B | NS | NS | |
| Chloroform | A | NS | A | |
| Ethyl acetate | A | NS | NS | |
| Chlorobenzene | B | NS | NS | |
| Tetrahydrofuran | A | NS | NS | |
| 1,4-Dioxane | A | NS | amorphous | |
| Isopropyl ether | B | NS | NS | |
| Toluene | B | NS | amorphous | |
| Cyclohexane | B | NS | NS | |
| Heptane | B | NS | NS | |
| 1-Butanol | A | FB | A | |
| 2-Propanol | A | low solids | amorphous | |
| Trifluoroethanol | NS | NS | B | |
| Dimethyl carbonate | A | NS | NS | |
| t-Butyl methyl ether | B | NS | NS | |
| Isopropyl acetate | B | NS | NS | |
| Ethanol | A | A | A | |
| 1-Methoxy-2-propanol | A | NS | A | |
| Cyclohexanone | A | NS | low solids | |
| N,N-Dimethylformamide | NS | NS | NS | |
| 2-Methoxyethyl ether | A | NS | NS | |
| Methanol:Water (95:5) | NS | NS | B | 0.20 |
| Acetonitrile:Water (95:5) | A | NS | amorphous | 0.60 |
| Acetone:Water (95:5) | A | NS | FB | 0.60 |
| Tetrahydrofuran:Water (95:5) | A | NS | amorphous | 0.82 |
| 2-Propanol:Water (95:5) | A | NS | amorphous | 0.55 |
| Methanol:Water (90:10) | NS | NS | B | 0.33 |
| Acetonitrile:Water (90:10) | A | NS | B | 0.76 |
| Acetone:Water (90:10) | A | NS | amorphous | 0.70 |
| Tetrahydrofuran:Water (90:10) | A | NS | amorphous | 0.83 |
| 1,4-Dioxane:Water (90:10) | A | NS | amorphous | 0.70 |
| 2-Propanol:Water (90:10) | A | NS | B | 0.65 |
| Acetone:Water (80:20) | A | NS | B | 0.77 |
| Ethanol:Water (20:80) | NS | NS | NS | 0.93 |
| 2-Propanol:Dimethylsulfoxide | NS | NS | A | |
| (80:20) | | | | |
| Acetonitrile:Dimethylsulfoxide | A | A | NS | |
| (80:20) | | | | |
| N-Methyl-2-pyrrolidone | NS | NS | NS | |

LEGEND:

| | |
|---|---|
| A | Form A |
| B | Form B |
| NS | no solid observed |
| FB | free base |

Characterization of Form A. Form A is a white powder and was determined to be crystalline by Raman (FIG. 1) and PXRD analysis (FIG. 2). DSC shows a broad, shallow endotherm from 25-150° C. followed by an endotherm occurring with decomposition at 216.4° C. (FIG. 3B). TGA-IR analysis showed release of 2.9% water from 25-150° C. (1 eq., monohydrate) that corresponds with the broad DSC endotherm (FIG. 3A).

Characterization of Form B. Form B is a hydrate form observed during the screen. Raman (FIG. 4) and PXRD (FIG. 5) analyses indicate Form B is crystalline. DSC shows a broad endotherm from 25-110° C. (FIG. 6B) that is associated with 9.4% weight loss of water (3.4 eq.) by TGA-IR (FIG. 6A). DSC analysis also shows a small, low energy, endotherm at 147.7° C. Form B was confirmed to be a di-HCl salt by IC.

### Example 3. Relative Stability Studies of Forms A and B

Relative stability studies were conducted at 25° C. on monohydrate Form A and hydrate Form B to determine the stable hydrate at various water activity levels.

Saturated suspensions of Form A were prepared by stirring excess Form A in the specified solvent system. The suspension was stirred overnight at 25° C. A clarifying filtration was performed and the filtrate was added to a 2 mL vial containing ˜10 mg of Form A and ˜10 mg of Form B. The resulting suspensions were stirred at 25° C. for seven days, isolated, dried under vacuum for 45 minutes, and analyzed by FT-Raman.

Form A was obtained after the ripening study. The summarized results of the study are shown in Table 3 and indicate Form A is the stable hydrate at 25° C. over the entire water activity range.

TABLE 3
Results of Competitive Ripening Study

| | Water | Temp | Final Form |
|---|---|---|---|
| Solvent (v/v) | Activity (a_{w}) | (° C.) | Observed |
| Ethanol | 0 | 25 | Form A |
| Methanol:Water | 0.5 | 25 | Form A |
| (73:27) | | | |
| Acetone:Water | 0.75 | 25 | Form A |
| (83:17) | | | |
| Water | 1 | 25 | Form A |

### Example 4. Additional Crystal Form Screen

### Instrumentation

High Resolution X-Ray Powder Diffraction (high resolution XRPD). High-resolution diagrams are recorded at ambient conditions on a Panalytical X'Pert Pro MPD powder diffractometer using the Bragg-Brentano (vertical θ-2θ configuration) parafocusing geometry coupled with a X'Celerator detector. A sealed copper anode X-ray tube is used, running at 45 kV and 40 mA levels. An incident beam monochromator (Johansson type: a symmetrically cut curved germanium (111) crystal) produces pure Cu K α 1 radiation (λ=1.54060 \acute{Å}). A thin layer of the product is deposited on a single-crystal silicon wafer, cut out according to Si (510) crystallographic orientation that, by systematic extinction, impedes any Bragg reflection. In order to bring more crystallites into the diffraction position and thus reduce the influence of particle statistics on the measurements, a sample spinner is used. The spinner rotation speed is set at 1 revolution per second. The angular range extends from 2 to 50° in 2θ, with a 0.017° step size in 2θ. A variable counting time from 500 to 5000 seconds per step was used.

X-Ray Powder Diffraction (XRPD). XRPD analyses are carried out on a Siemens-Bruker D5000 Matic powder diffractometer using the Bragg-Brentano (vertical θ-2θ configuration) parafocusing geometry. A sample feeder makes it possible to automate the work. If enough of the product is available, the powder is top-loaded on a concave stainless steel sample holder. Otherwise, a thin layer of the product is deposited on a single-crystalline silicon wafer, cut out according to Si (510) crystallographic orientation that, by systematic extinction, impedes any Bragg reflection. A sealed cobalt anode X-ray tube running at 40 kV and 30 mA levels is used. Two lines are typically emitted: CoKα1 (λ=1.7890 Å) and CoKα2 (λ=1.7929 Å). An Iron β-filter, placed between the detector and specimen, does not altogether eliminate CoKβ (λ=1.6208 Å) radiation, which still contributes about 1% of the diffracted beam at the detector (manufacturer's data). The primary beam passes through a parallel plate collimator (0.2 mm Soller slits), then through a divergence slit (0.2 mm). A Braun 50 M multicanal linear detector completes the setup. It has a 8°-wide detection window in angle 2θ. Diagrams should be recorded in the following conditions: a 2 to 50.0° scan in angle 2θ, 20 seconds counting time per degree in 2θ, and ambient conditions of pressure, temperature and relative humidity.

Temperature and Relative Humidity X-Ray powder diffraction. Tests are carried out with a Siemens-Bruker D5000 diffractometer equipped with the Bragg-Brentano parafocusing (θ-θ) geometry and an Anton-Paar TTK450 temperature chamber. For certain tests a dry nitrogen or RH streams is used. The powder is deposited in a concave stainless steel sample holder. A sealed cobalt anode X-ray tube running at 40 kV and 30 mA levels is used. Two lines are typically emitted: CoKα1 (λ=1.7890 A) and CoKα2 (λ=1.7929 Å). An Iron β-filter, placed between the detector and specimen, does not altogether eliminate CoKβ (λ=1.6208 Å) radiation, which still contributes about 1% of the diffracted beam at the detector (manufacturer's data). The beam is sighted using Soller slits, to improve its parallelism. Variable divergence slits keep the illumination area of the sample constant. A 1 mm collimator limits diffusion between the tube and the sample. A Braun 50-M multicanal linear detector completes the setup. It has a 8°-wide detection window in angle 2θ. Temperature is allowed to rise at a rate of 0.05° C./sec. Diagrams are usually recorded in the following conditions: a 1.5 to 50.0 degree scan in angle 2θ, 10 to 15 seconds counting time per degree in 2θ. Data are acquired in isotherm mode when the requested temperature is reached.

Simultaneous Thermogravimetric Analysis coupled FTIR spectrometer (TGA-FTIR). Analyses are carried out using a TG209C Netzsch Instrument coupled with a Tensor 27 Bruker FTIR spectrometer. This system allows simultaneous thermo-gravimetric analysis (TGA) and FTIR chemical identification of the evolved compounds (water and solvents). The evolved gases are carried off to the FTIR spectrometer through a transfer line heated to 476 K to prevent condensation of the evolved products. A sample mass of 5 to 10 mg is deposited in an aluminum crucible. The TGA-FTIR analysis is conducted under a dry nitrogen stream at 10 mL/min. Usually the sample is heated from 298 to 520-570 K at a rate of 5 K/min. A spectral domain from 4000 to 700 cm-1, a resolution of 4 cm-1 and 20 scans/spectrum are used for the FTIR spectrum recording. For each solvent to be analyzed by FTIR a specific wave number range related to the solvent type has to be chosen.

Thermogravimetric analysis. Analyses are carried out on a T.A. instruments TGAQ500 or TGAQ5000 analyzers. Mass calibration is performed with 10 and 100 mg certified masses and the instrument is temperature calibrated with alumel and nickel standards (Curie points of respectively 154° C. and 354° C.). Samples are exposed to a constant nitrogen stream of 60 mL/min and temperature ranges from 20 to 250° C. at a 5° C./min rate. The quantity of product lies between 2 and 5 mg. The powder is deposited in an open aluminum sample pan, which is itself placed in a platinum pan.

Differential Scanning calorimetry (DSC). Analyses are carried out under a nitrogen stream with a T.A. Instruments Q1000 (or a Q200) analyzers. The calorimeters are temperature-calibrated with indium and lead (onset temperatures of 156.6° C. and 327.5° C. respectively). Energy calibration is done with a certified indium calibrator (melting enthalpy of 28.45 J/g). A mechanical compressor is used to obtain and equilibrate the temperature program: from 0 to 270° C. at a rate of 5° C./min under a constant nitrogen stream of 55 mL/min (respectively 50 mL/min). The quantity of product analyzed lies between 1 and 5 mg, and is placed in a crimped or aluminum sample pan.

Moisture sorption/desorption isotherms. All experiments were performed on a DVS-1 automated gravimetric vapour sorption analyser (Surface Measurement Systems Ltd., London, UK). The DVS measures the uptake and loss of vapour gravimetrically using a Cahn D200 recording ultra-microbalance with a mass resolution of ±0.1 μg. A controlled relative humidity is generated by mixing different proportions of dry and water saturated carrier gas streams (monitored by mass flow controllers). The temperature was maintained constant, ±0.1° C., by enclosing the entire system in a temperature-controlled incubator. A sample size around 10 mg was used. Prior to being exposed to any water vapour the samples were dried at 0% relative humidity (RH) to remove any surface water present and establish a dry, baseline mass. Next, the samples were exposed to an increasing relative humidity raised by a step of 5% RH from 0% to 95% RH (or 90% RH). At each stage, the sample mass was allowed to reach equilibrium before the relative humidity was increased or decreased (considering that equilibrium was established when dm/dt ratio (m=mass; t=time) did not exceed the value of 3.3 10-4 mg/s during 30 minutes). If equilibrium state was not reached, the change in relative humidity took place automatically after 600 minutes. Two consecutive cycles were recorded. From the complete moisture sorption and desorption profile an isotherm was calculated using the DVS Advanced Analysis Suite v3.6. All experiments were performed at 25.0° C.

Polymorph screening was carried out varying solvents, supersaturation, temperature and water activity. Generally, the conditions used were:
crystallization by slow evaporation at room temperature, evaporation at high temperature under atmospheric pressure or vacuum, dissolution at reflux temperature followed by slow cooling at room temperature
precipitation by addition of non-solvents, solvent transfer by azeotropic distillation
slurrying of a pure form or a mixture of forms at room temperature in anhydrous solvents or aqueous solvents

The polymorphs identified by the crystallization studies using XRPD results are provided in Table 4:

TABLE 4
Results of Crystallization Studies

| Solvent or | | Crystalline Form |
|---|---|---|
| Solvent Mixture | Conditions | Observed |
| methanol | flash evaporation | amorphous |
| water | slow evaporation | Form B |
| methanol | slow evaporation | Form A |
| ethanol | slow evaporation | Form A |
| hexafluoroisopropanol | slow evaporation | Form H |
| acetonitrile | slow evaporation | mixture of forms |
| isopropanol | slow evaporation | Form E |
| n-butanol | slow evaporation | Form A |
| dimethylformamide | slow evaporation | Form A |
| water/butanone | distillation | Form A |
| water/methyl isobutyl | distillation | Form A |
| ketone | | |
| water/tetrahydrofuran | distillation | Form A |
| water/acetonitrile 20/80 | crystallization | Form A |
| ethanol | crystallization | Form I |
| water/acetone 50/50 | crystallization | Form A and Form B |
| water/acetone 20/80 | crystallization | Form A |
| water/acetonitrile 50/50 | crystallization | Form A |
| isopropanol/water 95/5 | crystallization | Form A |
| acetonitrile | crystallization by cooling | Form A |
| methanol | crystallization by cooling | Form A |
| ethanol | crystallization by cooling | Form A |
| n-propanol | crystallization by cooling | Form A |
| n-butanol | crystallization by cooling | Form A |
| isopropanol | crystallization by cooling | Form A |
| heptane (solution | precipitation | Form A |
| in methanol) | | |
| heptane (solution | precipitation | Form A |
| in methanol) | | |
| tetrahydrofuran | precipitation | Form A |
| (solution in water) | | |
| tetrahydrofuran | precipitation | Form A |
| (solution in water) | | |

### Example 5. Variable temperature T-XRPD analysis of Form A and Form B.

Crystal forms identified in Example 4 were characterized as follows.

The structural behavior of Form A under heating was studied in situ by T-XRPD under a nitrogen atmosphere. The temperature was raised by 10° C. steps from room temperature to 230° C. Structural modifications of the XRPD diagram due to heat are observed (FIG. 7). From 40° C. to 80° C. the emergence of Form C is recorded, corresponding to the dehydration form of Form A. Then between 80° C. to 90° C. a structural modification was observed. At 90° C. a new XRPD pattern appears, which corresponds to another anhydrous crystalline phase. Between 160° C. to 170° C. a major structural modification was observed.

The structural behavior of Form B under heating was studied in situ by T-XRPD under a nitrogen atmosphere. The temperature was raised by 10° C. steps from room temperature to 160° C. A modification of the XRPD diagram due to heat-induced loss of water molecule and dilation of the crystal lattice was observed from room temperature to 70° C., resulting in Form D (FIG. 8). Between 90° C. to 120° C. no structural modification was observed. Finally at 130° C., the XRPD diagram is flat and be similar to an amorphous material.

### Example 6. Characterization of Crystal Forms

Crystal forms identified in Example 4 were characterized as follows.

Form F. XRPD of the tetraisopropanol solvate is depicted in FIG. 10.

DVS of the tetraisopropanol solvate was carried out at 25° C. after “0% P/P₀” pre-treatment at 25° C. (FIG. 12). Once the sample is exposed to increasing partial pressure of isopropanol, a very slight and continuous isopropanol adsorption is measured between 0 and 45% P/P_{0 (IPA)} (2.1% at 45% P/P_{0 (IPA)}). Between 50 and 55% P/P_{0 (IPA)} an important isopropanol uptake is observed reaching 25%. Between 60% P/P_{0 (IPA)} and 90% P/P_{0 (IPA)} a continuous isopropanol uptake is observed reaching 3%. Between 90% P/P_{0 (IPA)} and 40% P/P_{0 (IPA)} a very slight and continuous isopropanol loss is observed reaching 3%. Afterwards between 35% P/P_{0 (IPA)} and 0% P/P_{0 (IPA)} a very important isopropanol loss is observed reaching 30%. XRPD of the tetraisopropanol solvate sample following DVS reveals a new form characterized by XRPD as a monoisopropanol solvate Form E (FIG. 9).

The TGA-IR curve of Form F is shown in FIG. 11. A first weight loss of 22% corresponding to 3 moles of isopropanol is recorded from room temperature to around 90° C. A second weight loss of 7% corresponding to 1 mole of isopropanol is recorded from 90° C. to around 160° C. Beyond 210° C., thermal decomposition is observed.

Form G. The XRPD pattern of the water:isopropanol heterosolvate is shown in FIG. 13.

The TGA-IR curve of the water:isopropanol heterosolvate is shown in FIG. 14. A first weight loss of 3% corresponding to water sorption is recorded from room temperature to around 100° C. A second weight loss of 5% corresponding to 0.5 mole of isopropanol is recorded from 100° C. to around 160° C. Beyond 210° C., thermal decomposition is observed.

DVS of the water:isopropanol heterosolvate is carried out at 25° C. after “0% RH” pretreatment at 25° C. (a partial dehydration and is observed with a pre-treatment at 25° C.) (FIG. 15). After one day of exposure at 25° C. at 0% RH, partial dehydration and desolvation of the sample is observed with the loss of 7.7% of water and IPA. Once the sample is exposed to increasing relative humidity, an important and continuous water adsorption is measured between 5 and 65% RH (7.4% at 65% RH). Between 70 and 90% RH a slight and continuous water uptake is observed reaching 0.7%. Between 90% RH and 10% RH a continuous water uptake is observed reaching 2.5%. At 10% RH, an important water loss is measured, reaching 7.1%. At 0% RH, a difference between the first and the second cycle is recorded (reaching 0.6%) that could correspond to a structural modification of the crystalline phase.

Form H. The XRPD pattern of the hexafluoroisopropanol solvate is shown in FIG. 16.

The TGA curve of the hexafluoroisopropanol solvate is shown in FIG. 17. A first weight loss of 14.8% is recorded from 60° C. to around 100° C. (likely corresponding to 0.5 mole of hexafluoroisopropanol, TGA-IR or TGA-MS measurements were not performed). A second weight loss of 8.5% is recorded from 100° C. to around 160° C. Beyond 180° C., thermal decomposition is observed.

DVS curves are carried out at 25° C. on hexafluoroisopropanol solvate (FIG. 18). After 6 hours under nitrogen a weight loss of 5% is recorded (a partial desolvation is observed with a pre-treatment at 25° C.). During the first sorption cycle, an important weight loss is recorded between 0 and 75% RH (around 30%). This weight loss corresponds to the structural modification indicative of desolvation with solvent exchange induced by water vapour. Form H is confirmed to transform into Form B.

Form I. The XRPD pattern of the ethanol solvate is shown in FIG. 19. Form I is an efflorescent solvate under ambient conditions.

1. A crystalline form of Compound 1:

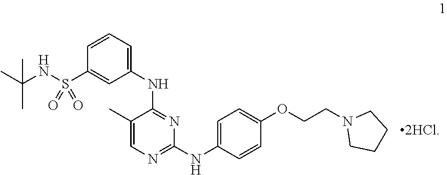 [SMILES: Cc1cnc(Nc2ccc(OCCN3CCCC3)cc2)nc1Nc1cccc(S(=O)(=O)NC(C)(C)C)c1]

2. The crystalline form of claim 1, wherein the form is a hydrate.

3. The crystalline form of claim 2, wherein the form is a monohydrate.

4. The crystalline form of claim 2, wherein the monohydrate is characterized by one or more peaks in its X-ray powder diffraction pattern selected from 4.3, 9.6, 10.0, 12.4, 12.7, and 17.0±0.2 degrees 2θ.

5. (canceled)

6. The crystalline form of claim 2, wherein the form is a trihydrate.

7. The crystalline form of claim 6, wherein the trihydrate is characterized by one or more peaks in its X-ray powder diffraction pattern selected from 5.4, 6.2, 11.6, 13.9, 16.4, and 16.7±0.2 degrees 2θ.

8. (canceled)

9. The crystalline form of claim 1, wherein the form is anhydrous.

10. The crystalline form of claim 9, wherein the anhydrous form is characterized by one or more peaks in its X-ray powder diffraction pattern selected from 4.3, 6.2, 8.6, 9.7, 13.6, and 17.3±0.2 degrees 2θ.

11. (canceled)

12. The crystalline form of claim 9, wherein the anhydrous form is characterized by one or more peaks in its X-ray powder diffraction pattern selected from 12.8, 13.6, 14.9, 16.1, and 17.2±0.2 degrees 2θ.

13. (canceled)

14. The crystalline form of claim 1, wherein the form is a solvate.

15. The crystalline form of claim 14, wherein the form is a monoisopropanol solvate.

16. The crystalline form of claim 15, wherein the monoisopropanol solvate is characterized by one or more peaks in its X-ray powder diffraction pattern selected from 7.2, 10.4, 10.8, 13.2, and 17.5±0.2 degrees 2θ.

17. (canceled)

18. The crystalline form of claim 14, wherein the form is a tetraisopropanol solvate.

19. The crystalline form of claim 18, wherein the tetraisopropanol solvate is characterized by one or more peaks in its X-ray powder diffraction pattern selected from 5.3, 6.4, 8.2, 10.5, 15.3, and 15.7±0.2 degrees 2θ.

20. (canceled)

21. The crystalline form of claim 1, wherein the form is a heterosolvate.

22. The crystalline form of claim 21, wherein the form is a water-isopropanol heterosolvate.

23. The crystalline form of claim 22, wherein the water-isopropanol heterosolvate is characterized by one or more peaks in its X-ray powder diffraction pattern selected from 7.3, 7.6, 10.4, 10.8, and 17.5±0.2 degrees 2θ.

24. (canceled)

25. The crystalline form of claim 14, wherein the form is a hexafluoroisopropanol solvate.

26. The crystalline form of claim 25, wherein the hexafluoroisopropanol solvate is characterized by one or more peaks in its X-ray powder diffraction pattern selected from 4.3, 6.0, 6.9, 10.9, 11.5, 14.7, and 17.1±0.2 degrees 2θ.

27. (canceled)

28. The crystalline form of claim 14, wherein the form is an ethanol solvate.

29. The crystalline form of claim 28, wherein the ethanol solvate is characterized by one or more peaks in its X-ray powder diffraction pattern selected from 5.3, 10.6, and 15.9±0.2 degrees 2θ.

30. (canceled)

31. A sample comprising the crystalline form of claim 1, wherein the sample is substantially free of impurities.

32. The sample of claim 31, wherein the sample comprises at least about 90% by weight of Compound 1.

33-34. (canceled)

35. The sample of claim 31, wherein the sample comprises no more than about 5.0 percent of total organic impurities.

36-39. (canceled)

40. A method of inhibiting activity of a JAK2 kinase, or a mutant thereof, in a biological sample or a patient comprising the step of contacting said biological sample with a crystalline form of claim 1, or a composition thereof.

41. (canceled)

42. A method for treating a JAK2-mediated disease or disorder, in a patient in need thereof, comprising the step of administering to the patient a crystalline form of claim 1, or pharmaceutically acceptable composition thereof.
